(19) United States
(12) Patent Application Publication
(10) Pub. No.: US 2010/0016935 A1
(43) Pub. Date: Jan. 21, 2010

(54) MEDICAL IMPLANTABLE LEAD

(76) Inventors: Hans Strandberg, Sundbyberg (SE); Anna Norlin-Weissenrieder, Stockholm (SE); Leda Henriquez, Bandhagen (SE); Eva Harström, Hässelby (SE); Mikael Sjögren, Fjardhundra (SE); Annika Naeslund, Bromma (SE)

Correspondence Address: SCHIFF HARDIN, LLP;PATENT DEPARTMENT, 233 S. Wacker Drive-Suite 6600, CHICAGO, IL, 60606-6473, US

(21) Appl. No.: 12/447,021

(22) PCT Filed: Oct. 25, 2006
(86) PCT No.: PCT/SE06/01209

§ 371 Date: Apr. 24, 2009

(51) Int. Cl. A61N 1/05; H05K 13/00

(52) U.S. Cl. 607/116; 29/829

(57) ABSTRACT

A implantable lead an elongate body including a flexible insulating tube, and a tubular conductor layer formed of multiple separate strip conductors, which are arranged at the outer surface of the insulating tube and extend along the length thereof.

### BACKGROUND OF THE INVENTION

1. Field of the Invention

The present invention relates to a medical implantable lead comprising an elongate body including a flexible insulating tube and a plurality of conductors.

2. Description of the Prior Art

A medical implantable lead is preferably designed as thin as possible. It also needs to be well flexible in order to be able to follow narrow winding body cavities. A conventional structure is an elongate lumen defined, i.e. formed, by coiled conductors carrying electrical signals for different applications. The lumen is used for facilitating implantation of the flexible lead into a body by means of a slightly stiffer guide wire, stylet or the like, which is inserted into the lumen and maneuvered by an operator, typically a surgeon.

Modern technology imposes demands on increased ability to carry more and more signals for sensing, monitoring and commanding purposes. These demands introduce a conflict between outer diameter of the lead and number of available conductors within the lead, since with the traditional design of the lead a coaxial addition of a conductor coil adds significantly to the diameter of the lead.

Therefore different ways to increase the number of conductors without increasing the outer dimensions of the lead have been proposed. For example, in U.S. Pat. No. 5,201,903 to Corbett et al. there is shown a multi conductor electrical cable, which is said to be suitable for implantation in living bodies. The main embodiment has several, e.g. seven, separately insulated conductors, helically twinned to a cable, which is provided with a further insulating coating forming a single, or integral, unit. The conductors are thin wire conductors having diameters as small as about ten micrometers. There is no teaching in U.S. Pat. No. 5,201,903 of how to implant such a cable into the body. A central lumen is disclosed, which is meant to be used as a catheter, however being to thin to work as a lumen for a stylet or the like. However, it would probably be a simple task to enlarge the central lumen. Notwithstanding the positive properties of such a thin multi conductor cable, it is also suffering from some disadvantages. The manufacturing process of handling such thin wire conductors and embed them in an insulating material to form the electrical cable is rather a difficult task.

### SUMMARY OF THE INVENTION

It is an object of the present invention to provide a medical implantable lead that alleviates the above-mentioned drawbacks of the prior art.

This object is achieved by a medical implantable lead according to the present invention

Thus, in accordance with an aspect of the present invention, a medical implantable lead has an elongate body including a flexible insulating tube, and a tubular conductor layer consisting of a plurality of separate strip conductors, which are arranged at the outer surface of the insulating tube and extend along the length thereof.

In accordance with another aspect of the present invention the above object is achieved by a method for manufacturing a medical implantable lead, including the steps of providing a flexible insulating tube, and providing the insulating tube with a conductor layer, including a number of separate strip conductors extending along the insulating tube, at an outer surface thereof.

Thus, in its simplest form a tube provided with conductors on the outer surface thereof is obtained in accordance with the teaching of the present invention. Since the tube is flexible, by inserting a stylet or the like into the central cavity thereof, it is possible to implant the lead into a body cavity guided/controlled by means of the stylet. The application of strip conductors, which per se are very thin, and optionally wide, makes it possible to easily arrange a large number of conductors side by side around the tube.

According to an embodiment of the medical implantable lead the elongate body has an insulating layer, which is arranged coaxially of the insulating tube and which covers the strip conductors. This is a typical structure for intra body applications, where the conductors should be insulated from the ambient environment as well as from each others.

According to another embodiment of the medical implantable lead the strip conductors and composed of metal, which has been deposited on the insulating tube. Several techniques already in use are applicable for forming the strip conductors on the insulating tube by means of depositing the metal, for example sputtering, vapor deposition, deposition from a liquid solution, etc.

According to another embodiment of the medical implantable lead the elongate body has a further insulating tube arranged coaxially of the insulating tube and enclosing the multiple strip conductors. Thus, the insulating layer can be of different kinds, such as another tube similar to the basic one.

According to another embodiment of the medical implantable lead the elongate body comprises a further tubular conductor layer of one or more strip conductors arranged on the outer surface of the further insulating tube. In other words, it is possible to form a lead that has two, or more, conductor layers, which are coaxially arranged with insulating material between the conductor layers. A conductor layer can be a single conductor forming a thin metal tube or a portion of a tube, or a large number of stripes arranged at a fraction of the circumference from each other.

These and other aspects, features, and advantages of the invention will be apparent from and elucidated with reference to the embodiments described hereinafter.

### BRIEF DESCRIPTION OF THE DRAWINGS

FIG. 1 is a side view of an embodiment of a medical implantable lead according to the present invention.

FIG. 2 is an enlarged perspective view of a portion of the lead shown in FIG. 1.

FIGS. 3-5 are cross-sectional views of different embodiments of leads according to the present invention.

FIGS. 6a and 6b are enlarged views of a connector portion of a lead shown in FIG. 1, wherein FIG. 6a is a partially X-ray view and FIG. 6b is a partially cut away view.

### DESCRIPTION OF THE PREFERRED EMBODIMENTS

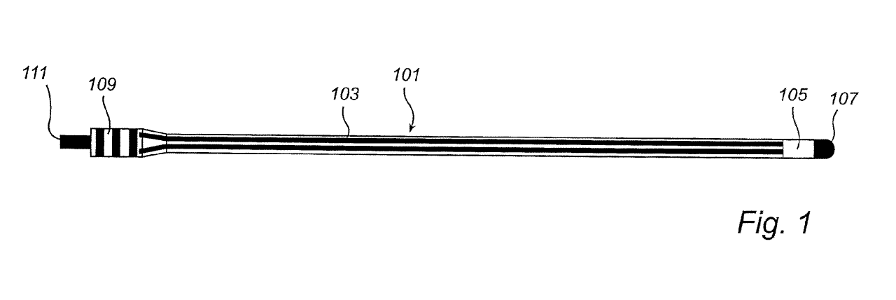

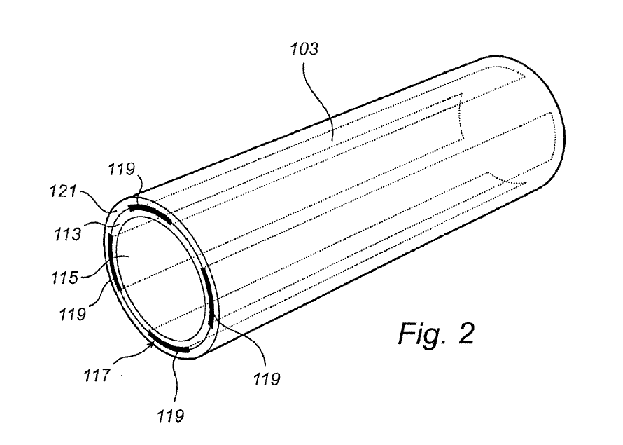

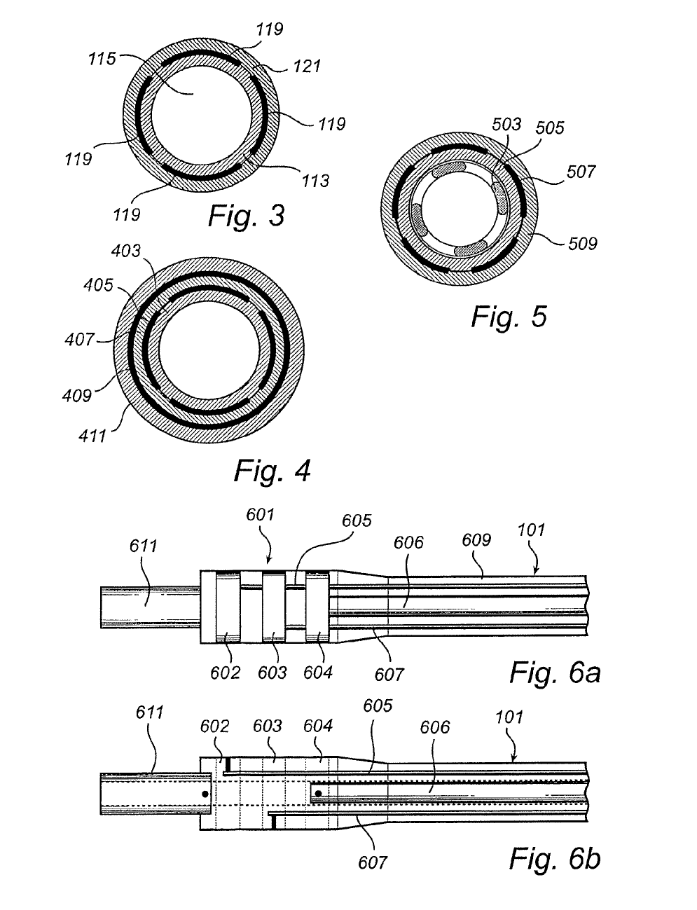

A first embodiment of a lead 101 according to this invention has an elongate body 103, an electrode tip 105 at a distal end 107 of the lead 101, and a connector, or connector portion, 109 at a proximal end 111 of the lead 101. The lead 101, and more particularly the elongate body 103, further has a first, or inner, insulating tube 113, which has a central cavity 115, as shown in FIG. 2, a conductor layer 117 composed of four conductors 119, which are arranged on the outer surface of the first insulating tube 113, and a second, or outer, insulating tube 121, which is arranged coaxially of the first tube 113 and outside of the conductor layer 117, and covers the conductors 119 in order to protect them from the ambient environment and from short circuits between the conductors 119. The conductors 119, two of which can also be seen in FIG. 1 through the outer insulation layer, are strip shaped and extend in parallel along the length of the lead 101 from the connector 109 to the electrode tip 105. Thus the conductors 119 can be considered to be sectorially arranged around the elongate body 103. The structure of the elongate body is even clearer from the cross-sectional view of FIG. 3.

Since the strip conductors are very thin, and the insulating layers are also thin, it is possible to construct various combinations of conductor layers and insulating layers. One such combination, as shown in FIG. 4, has an inner insulating tube 403, a first layer of conductors 405 arranged on the inner tube 403, a middle insulating tube 407 covering the first conductor layer 405, a second conductor layer 409 arranged on the middle insulating tube 407, and an outer insulating tube 411 covering the second conductor layer 409. The layers are, thus, all arranged coaxially having a common central longitudinal axis. In this embodiment, the first conductor layer 405 consist of four conductors, while the second conductor layer 409 is composed of a single conductor forming a tube. Such a larger area conductor 409 is useful for carrying the largest current that is required, such as stimuli pulses for pacing a heart. Simultaneously the other conductors 405 can be used for sensor signals from sensors at the electrode tip, control signals to a device at the distal end of the lead, etc.

It is also possible to combine this lead design with the conventional coil conductors, as shown in FIG. 5. As an innermost tube a conducting coil 503 made up of four spiralled filaments is provided. Then, proceeding radially away from the centre of the lead an insulating tube 505, a conductor layer 507 and an outer insulating tube 509 are provided, in that order.

In order to facilitate connection of the elongate body 103 to devices and electrode tips, or bodies, in one embodiment of the lead a connector portion, or connector, 109 is formed at each end of the elongate body 103. The connector at the proximal end 111 is illustrated more closely in FIGS. 6a and 6b. The connector 601 has three circumferential connection rings 602-604, which are arranged consecutively at a small distance from each other along a portion of the lead 101 close to its proximal end. Each ring 602-604 is connected radially of the lead towards the centre thereof with strip conductor 605-607 positioned beneath the ring. That is, each conductor 605-607 has a radially extending end portion, which extends passed the outer insulating tube 609 of the lead 101 to the respective ring 602-604, with which it is connected. However, there is a fourth strip conductor, which is connected radially inwards with a hollow central pin 611 of the connector 601. Thus, the central stylet lumen extends through the connector pin 611, as shown with broken lines in FIG. 6b. The lead 101 can be provided with a similar connector at the distal end 107 thereof, which connector is then connected with the electrode tip 105.

The lead 101 is manufactured as follows. An insulating tube of a suitable plastic or rubber material is formed. The tube is then used as a substrate upon which the conductors are formed. Thus, a thin layer of metal is formed on the lateral area of the tube by means of a suitable method. Preferably an epitaxial process is employed. For example the metal is deposited by sputtering, i.e. using a metal plasma in vacuum, or at a low pressure, by chemical deposition, i.e. a chemical reduction of metal salts in a water solution causes a deposition on an available surface, or by chemical vapor decomposition, i.e. a gas comprising metal compositions is decomposed and then the metal is deposited on an available surface. In order to form a number of separate stripe conductors, a protective film, e.g. a photoresist, is applied to the metal layer, and photo hardened through a mask providing a desired pattern of stripes. Unprotected areas are then etched off. An insulating layer is then applied upon the conductor layer. This insulating layer can be anything from a thin cover to a thicker one having about the same thickness as the innermost tube. Further layers of conductors and insulating material can then be applied in further coaxial tubular structures.

Above, embodiments of the lead and the method for manufacturing a lead according to the present invention have been described. These should be seen as merely non-limiting examples. As understood by those skilled in the art, many modifications and alternative embodiments are possible within the scope of the invention as defined by the appended claims.

Although modifications and changes may be suggested by those skilled in the art, it is the intention of the inventors to embody within the patent warranted hereon all changes and modifications as reasonably and properly come within the scope of their contribution to the art.

We claim as our invention:

1. A medical implantable lead comprising an elongate body including a flexible insulating tube (113), and a tubular conductor layer comprising a plurality of separate strip conductors (119), arranged at the outer surface of said insulating tube and extending along the length thereof.

2. A medical implantable lead according to claim 1, wherein said elongate body comprises an insulating layer arranged coaxially of said insulating tube and covering said plurality of strip conductors.

3. A medical implantable lead according to claim 1, wherein said plurality of strip conductors (119) consisting of deposited metal.

4. A medical implantable lead according to according to claim 1, wherein the elongate body comprises a further insulating tube arranged coaxially of said insulating tube and enclosing said plurality of strip conductors.

5. A medical implantable lead according to claim 4, wherein said elongate body comprises a further tubular conductor layer comprising at least one strip conductor arranged on the outer surface of said further insulating tube.

6. A method for manufacturing a medical implantable lead, comprising:
providing a flexible insulating tube;
providing said insulating tube with a conductor layer, including a plurality of separate strip conductors extending along the insulating tube, at an outer surface thereof.

7. A method according to claim 6, further comprising covering said conductor layer with a tubular insulating layer.

8. A method according to claim 6, wherein comprising, providing said insulating tube with a conductor layer by depositing a metal on said insulating tube.

9. A method according to claim 8, comprising depositing said metal by vapor deposition.
